(19) United States
(12) Patent Application Publication
(10) Pub. No.: US 2026/0285797 A1
(43) Pub. Date: Sep. 24, 2026

(54) PROCESS FOR PRODUCING BIOBASED a,B-UNSATURATED CARBOXYLIC ACIDS FROM POLY(3-HYDROXYALKANOATE)

(71) Applicant: Arkema France, Puteaux (FR)

(72) Inventors: Serge TRETJAK, Saint Avold Cedex (FR); Yves CABON, Saint Avold Cedex (FR)

(21) Appl. No.: 19/471,868

(22) PCT Filed: Mar. 29, 2024
(86) PCT No.: PCT/FR2024/050413

§ 371 Date: Oct. 2, 2025

(30) Foreign Application Priority Data
Apr. 5, 2023 (FR) FR23.03388

(51) Int. Cl. C07C 51/44

(52) U.S. Cl. CPC C07C 51/44

(57) ABSTRACT

The present invention relates to a process for manufacturing biobased α,β-unsaturated carboxylic acids from biomass containing a poly(3-hydroxyalkanoate), or from a solid poly(3-hydroxyalkanoate) previously extracted from said biomass in the presence of polymerization inhibitors, by thermolysis of said polymer, in the absence of a catalyst, predominantly into α,β-unsaturated carboxylic acid in gaseous form, without using an inert gas to facilitate the evaporation of this acid, This invention more precisely describes the step of thermolysis of the biomass or of the P3HA, followed by the purification steps affording the α,β-unsaturated carboxylic acid and recycling of the intermediate products.

### TECHNICAL FIELD

The present invention relates to a process for manufacturing biobased α,β-unsaturated carboxylic acids from biomass containing a poly(3-hydroxyalkanoate), or from a solid poly(3-hydroxyalkanoate) previously extracted from said biomass in the presence of polymerization inhibitors, by thermolysis of said polymer, in the absence of a catalyst, predominantly into α,β-unsaturated carboxylic acid in gaseous form, without using an inert gas to facilitate the evaporation of this acid. This invention more precisely describes the step of thermolysis of the biomass or of the P3HA, followed by the purification steps affording the α,β-unsaturated carboxylic acid and recycling of the intermediate products.

### PRIOR ART AND TECHNICAL PROBLEM

α,β-Unsaturated carboxylic acids are nowadays produced industrially mainly from feedstocks of fossil origin. For example, acrylic acid is obtained by oxidation of propylene, or methacrylic acid can be obtained by oxidation of isobutylene.

One possible way of obtaining these α,β-unsaturated carboxylic acids is the thermolysis at temperatures of 150 to 300° C. of the corresponding poly(3-hydroxyalkanoate) (P3HA), according to the following reaction:

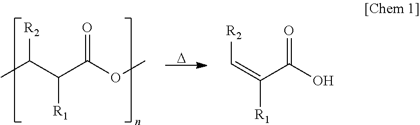

R₁═H or alkyl and R₂═H or alkyl; n is a number greater than 30

If R₁═R₂═H:
Poly(3-hydroxyalkanoate)=poly(3-hydroxypropionate) (P3HP);
α,β-Unsaturated carboxylic acid=propenoic acid (acrylic acid).

If R₁=methyl and R₂═H:
Poly(3-hydroxyalkanoate)=poly(3-hydroxyisobutyrate) (P3HiB);
α,β-Unsaturated carboxylic acid=isobutenoic acid (methacrylic acid).

If R₁═H and R₂=methyl:
Poly(3-hydroxyalkanoate)=poly(3-hydroxybutyrate) (P3HB);
α,β-Unsaturated carboxylic acid=but-2-enoic acid (crotonic acid).

If R₁═H and R₂=ethyl:
poly(3-hydroxyalkanoate) is poly(3-hydroxyvalerate) (P3HV);
α,β-Unsaturated carboxylic=pent-2-enoic acid

These poly(3-hydroxyalkanoates) can themselves be obtained beforehand by chemical transformations of raw materials of fossil origin, but also by fermentation of biomass.

There is strong market demand for these α,β-unsaturated carboxylic acids, which are used as monomers in numerous applications, to be obtained from biobased feedstocks. These biobased feedstocks are derived from renewable organic matter (biomass) of biological origin (microorganisms, plants or animals).

Document U.S. Pat. No. 9,850,192 describes a process for producing acrylic acid from a genetically modified microbial biomass metabolizing glucose or any other renewable feedstock to produce a poly-3-hydroxypropionate (P3HP) homopolymer or copolymer inside microbial cells. Said process comprises a step of thermolysis of the washed/dried/shredded P3HP-containing biomass in the presence of a catalyst. Acrylic acid is recovered in gaseous form and then condensed, while the catalyst and the residual mass of biomass can either be recycled into the process or subjected to thermal regeneration. However, there is a risk that the residue present in the reactor after thermolysis will be pasty and tacky, which could complicate its transfer to an industrial scale. Example 5 and FIG. 7 describe how to perform this invention on an industrial scale. After fermentation, the biomass is washed, and dried using either an atomizer or a twin-drum dryer. After addition of the catalyst, the product is pyrolyzed in a FAST™ reactor at 250° C.-350° C. with a residence time of between 0.25-1 hour, using an inert gas such as nitrogen to send the vapors formed to the purification equipment. The vapor phase is composed of 90% organic/water and 10% inert gas. The gas is then purified, according to the process described in U.S. Pat. No. 6,646,161 or in US 2012/0006673, to obtain acrylic acid still containing many impurities. Total purification is performed using distillation columns, as described in U.S. Pat. Nos. 7,332,624 and 7,179,875, and may also require crystallization operations, as described in U.S. Pat. Nos. 6,482,981 and 7,179,875. Said documents describe techniques usually used for obtaining acrylic acid via propylene oxidation.

US 2014/0018574 describes a process for the thermolysis of P3HA in the presence of a compound comprising a tertiary amine which acts as a catalyst, present in a concentration from about 0.5% to 4% by weight relative to the P3HA. Optionally, one or more inhibitors may be added to the P3HA before thermolysis is performed, preferably in a proportion of 150 ppm to 350 ppm relative to the P3HA, in the presence of depleted air (<6% 02) (0107-0108). Moreover, as regards the acrylic acid formed during this thermolysis, no inhibitor (0111) is added during its condensation. Finally, said document shows that at a temperature of 175° C. (00143) and in the absence of a catalyst, thermolysis was inoperative.

When the thermolysis of P3HA is performed in the liquid phase, as in JP 2015/067567, in the absence or presence of a catalyst and in the absence of an inhibitor, this reaction is preferably performed in the presence of an inert gas to evaporate the acrylic acid formed (0031).

Another solution consists in first extracting the P3HA from the biomass using an organic solvent before subjecting it to thermolysis. Example 6 of US 2015/0376152 describes the extraction of P3HP from biomass using an organic solvent, such as 2-butanone, followed by the production of acrylic acid in three steps: evaporation of the solvent and condensation of same in a receiving pot; thermal degradation of the P3HP in the absence of inhibitor, leading to the production of acrylic acid vapor, and finally distillation and condensation of the acrylic acid in a receiving pot containing hydroquinone to prevent polymerization of the acrylic acid.

In its patent application FR 2 208 914, the Applicant company proposes to perform thermolysis of the P3HA in the absence of a catalyst and in the presence of a polymerization inhibitor; characteristically, the vapor pressure of at least one of the inhibitors at the thermolysis temperature is at least twice the pressure at which thermolysis is performed, thus preventing the formation of polymers in the reactor and also in the gas phase in the event of accidental condensation or when the acrylic acid vapors condense at the top of the column.

In its patent application FR 2 208 916, the Applicant company describes a process using a solvent that allows P3HA to be selectively solubilized from biomass, separating out the insoluble organic detritus from said solvent, to perform a liquid-phase thermolysis treatment in the presence of polymerization inhibitors.

WO 2016/039618 describes the thermal degradation of a dry biomass containing poly(3-hydroxybutyrate) in order to produce crotonic acid. In Example 1, it is shown that crotonic acid can be obtained in comparable yields of less than 60% from wet or dry biomass, in the absence of a catalyst.

It has now been discovered that high-purity acrylic acid can be obtained by combining thermolysis of biomass containing P3HA, or thermolysis of solid P3HA and at least one polymerization inhibitor, with a staged thermolysis gas condensation system, fractional distillation and optionally crystallization, in the absence of a catalyst and without using an inert gas to promote evaporation of the α,β-unsaturated carboxylic acid formed.

More precisely, the term “in the absence of catalyst” means that the thermolysis of PHA in the presence of at least one polymerization inhibitor takes place in the absence of any other chemical species which accelerates or redirects the reaction kinetics. The thermolysis as envisaged is induced only by operating conditions such as temperature, pressure and residence time.

Chemical species which lead to different types of catalysis depending on the nature of the species are thus excluded:
homogeneous catalysis, if the catalyst and reagents only form a single (often liquid) phase;
heterogeneous catalysis, if the catalyst and reagents form several phases (generally a solid catalyst for reagents in the gas or liquid phase);
enzymatic catalysis, if the catalyst is an enzyme, i.e. a protein.

The term “in the absence of inert gas” means that the thermolysis of PHA in the presence of at least one polymerization inhibitor takes place in the absence of injection of a gas, for instance depleted air (<6% 02), which facilitates the evaporation of the acid formed and also the activation of the inhibitors in the reaction medium.

Consequently, the invention proposes to provide a simple and readily implementable solution affording α,β-unsaturated carboxylic acids from biomass containing P3HA, or from solid PH3HA, by using a thermolysis reactor coupled with an optimized purification process.

### SUMMARY OF THE INVENTION

A subject of the present invention is a process for manufacturing a biobased α,β-unsaturated carboxylic acid, said process comprising the following steps:
mixing a biomass containing a poly(3-hydroxyalkanoate) (P3HA), or solid P3HA, with at least one polymerization inhibitor in the absence of a catalyst;
subjecting said biomass/inhibitor or P3HA/inhibitor mixture to a thermolysis step, resulting in, firstly, said α,β-unsaturated carboxylic acid in the vapor phase and, secondly, a molten or solid residue, without addition of an inert gas;
condensing the gases resulting from thermolysis in one or more steps, and then feeding the resulting thermolysis gases to a distillation column,
fractionating the thermolysis gases into upgraded light products, heavy products recycled to the thermolysis reactor, and α,β-unsaturated carboxylic acid with a purity of greater than 98%, which can be crystallized to reach a purity >99.5%,
treating the solid-phase residue.

According to various embodiments, said process comprises the following features, where appropriate in combination. The contents indicated are expressed by weight, unless otherwise mentioned. In the stated ranges of values, the limits are included.

The term “thermolysis” of poly(3-hydroxyalkanoate) (P3HA) means its chemical decomposition into α,β-unsaturated carboxylic acid that occurs under the effect of temperature. This term is synonymous with pyrolysis.

According to one embodiment, the poly(3-hydroxyalkanoate) used in the process comprises a single type of 3-hydroxyalkanoate unit and the product formed is thus composed of a single a, β-unsaturated carboxylic acid.

According to one embodiment, the poly(3-hydroxyalkanoate) is poly(3-hydroxypropionate) and the α,β-unsaturated carboxylic acid produced is acrylic acid.

According to one embodiment, the poly(3-hydroxyalkanoate) is poly(3-hydroxyisobutyrate) and the α,β-unsaturated carboxylic acid produced is methacrylic acid.

According to one embodiment, the poly(3-hydroxyalkanoate) is poly(3-hydroxybutyrate) and the α,β-unsaturated carboxylic acid produced is crotonic acid.

According to one embodiment, the poly(3-hydroxyalkanoate) used in the process comprises a plurality of different 3-hydroxyalkanoate units and the product formed is therefore composed of a mixture of different α,β-unsaturated carboxylic acids. Examples of P3HA copolymers are poly-3-hydroxybutyrate-co-3-hydroxypropionate (poly-3HB-co-3HP) or poly-3-hydroxybutyrate-co-3-hydroxyvalerate (poly-3HB-co-3HV).

According to one embodiment, the poly(3-hydroxyalkanoate) contains the 3-hydroxypropionate unit and at least one of the α,β-unsaturated carboxylic acids produced is acrylic acid.

According to one embodiment, the poly(3-hydroxyalkanoate) contains the 3-hydroxyisobutyrate unit and at least one of the α,β-unsaturated carboxylic acids produced is methacrylic acid.

According to one embodiment, the poly(3-hydroxyalkanoate) contains the 3-hydroxybutyrate unit and at least one of the α,β-unsaturated carboxylic acids produced is crotonic acid.

According to one embodiment, the biomass host is a bacterium, yeast, fungus, alga, cyanobacterium or a mixture of two or more of these elements.

According to one embodiment, the biomass used is pretreated by washing, drying and shredding operations, resulting in a biomass containing from 30% to 90% by weight of PHA, with the remainder consisting of the cell membrane.

According to one embodiment, the biomass is subjected to a thermolysis reaction, which takes place in the presence of one or more polymerization inhibitors.

According to one embodiment, the P3HA with a purity of greater than 95% by weight, used in the process according to the invention, is obtained by extracting the P3HA with a solvent, evaporating off the solvent and removing the cell membrane by filtration and centrifugation.

According to one embodiment, the thermolysis reaction takes place in the presence of one or more polymerization inhibitors.

According to one embodiment, the mixing and thermolysis are performed continuously in successive or simultaneous steps.

According to one embodiment, the mixture comprising biomass comprises at least 0.01% of one or more polymerization inhibitors, up to 5% and preferably less than 1% of one or more polymerization inhibitors (mass contents).

According to one embodiment, the mixture comprising P3HA comprises at least 0.5% of one or more polymerization inhibitors, up to 90% of one or more polymerization inhibitors (mass contents) and preferably between 20% and 70% of one or more polymerization inhibitors.

According to one embodiment, the process according to the invention comprises one or more steps of condensation of the vapors of the α-β-unsaturated carboxylic acid(s) obtained via the poly(3-hydroxyalkanoate) thermolysis reaction.

According to one embodiment, the condensates obtained may be recycled into the thermolysis reactor and into the separation column.

According to one embodiment, one or more inhibitors are also injected into the condensers.

According to one embodiment, no inhibitors are injected into the condensers.

According to one embodiment, one or more distillation columns may be used to purify the α,β-unsaturated carboxylic acid.

According to one embodiment, the condensates obtained may be subjected to a rectification or crystallization treatment before being recycled into the thermolysis reactor.

According to one embodiment, the separation column is fed in the gas phase.

According to one embodiment, the distillation column tailstock is recycled into the thermolysis reactor.

According to one embodiment, the distillation column tailstock is subjected to a rectification or crystallization operation before being recycled into the thermolysis reactor.

According to one embodiment, the α,β-unsaturated carboxylic acid obtained at the top or from a side withdrawal has a purity of at least 98% by weight.

According to one embodiment, this α,β-unsaturated carboxylic acid may also be purified in a subsequent crystallization step.

According to one embodiment, the purity of the α,β-unsaturated carboxylic acid after crystallization is greater than 99.5% by weight.

According to one embodiment, the column head products are sent to a biological purification plant.

According to one embodiment, these column head products are upgraded to methane by hydrothermal gasification.

According to one embodiment, the process according to the invention comprises a step of treating the solid or molten residue at the end of thermolysis by upgrading it via recycling upstream of the thermolysis reactor or via an external treatment such as gasification.

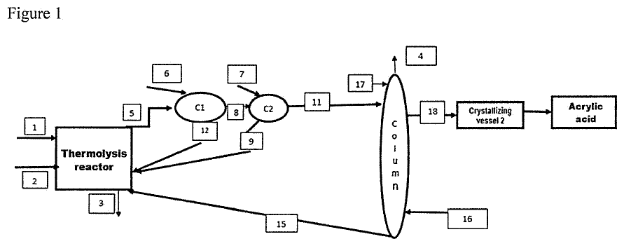

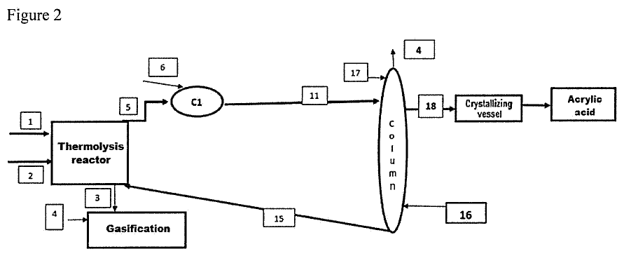

Other features and advantages of the invention will emerge more clearly on reading the detailed description which follows, with reference to the appended FIGS. 1 and 2.

FIG. 1 represents the block diagram of a facility for performing the process according to the invention, when it is applied to a P3HA/inhibitor mixture.

FIG. 2 represents the block diagram of a facility for performing the process according to the invention, when it is applied to a biomass/inhibitor mixture.

### DETAILED DESCRIPTION OF THE INVENTION

By way of example, the invention will be illustrated by describing the process applied to poly(3-hydroxypropionate) (P3HP) extracted from cell membrane, or to biomass still containing P3HP, making it possible to obtain α,β-unsaturated carboxylic acid (in this case acrylic acid) with a purity greater than 99.5% by weight.

The invention is directed toward the industrial-scale production of acrylic acid by thermolysis of poly(3-hydroxypropionate), limiting the problems of fouling of the thermolysis reactor and allowing this acid to be obtained with a purity >98% or even >99.5% by weight.

According to one embodiment, said process for producing biobased acrylic acid comprises the following steps, which may be performed sequentially or simultaneously:
Introduction of biomass (in powder form) or P3HP and at least one polymerization inhibitor, in the absence of catalyst, into a mixer (as solid phase) by means of a pipe or by an endless screw-type conveyor.
Mixing of the P3HP or biomass and at least one polymerization inhibitor in a conveyor mixer comprising several endless screws actuated in a sheath or directly in a reactor known as a thermolysis reactor.
Thermolysis of this mixture at a given temperature and controlled pressure in a system suitable for treating the molten or pasty residue, so as to generate a vapor phase and a viscous or even solid phase without injection of an inert gas.
Separation of the two phases formed in a gas/liquid separator.
Treatment of the residue for upgrading by land spreading, combustion, hydrothermal gasification or recycling upstream of the thermolysis reactor.
Staged condensation of the gas phase, performed by adjusting successive condensation temperatures using one or more condensers in series, and separation of the obtained gas and liquid phases containing acrylic acid and contaminants, which may be recycled back into the reactor or sent to the purification system.
Treatment of the condensed phase to obtain acrylic acid by using one or more distillation columns allowing, on the one hand, the separation of the acrylic acid from the products heavier than same and, on the other hand, the production of products lighter than same.
Purification of the acrylic acid obtained by a liquid/solid separation method such as crystallization, or by a gas/liquid separation method such as distillation.

The invention is based on the use of a biomass mixture containing a P3HA, or solid P3HA, and at least one polymerization inhibitor by performing a technique for mixing solids and heat treating this mixture.

The term “biomass” means organic matter originating from plants (including microalgae), animals, bacteria or fungi that may be used as a source of biobased feedstocks, as opposed to feedstocks of fossil origin.

In the process according to the invention, the first step uses genetically modified host biomass derived from genetic engineering. According to one embodiment, the biomass host is a bacterium, yeast, fungus, alga, cyanobacterium or a mixture of two or more of these elements.

The biomass is obtained by a prior step of culturing a recombinant host with a renewable feedstock. According to one embodiment, the renewable feedstock is chosen from glucose, fructose, sucrose, arabinose, maltose, lactose, xylose, ethanol, methanol, glycerol, fatty acids, plant oils and biomass-derived synthesis gas or a combination thereof.

According to one embodiment, the biomass used in the process according to the invention comes from a process of bacterial fermentation of sugars or lipids.

Depending on the culture conditions and on the variety of microorganism used, poly(3-hydroxyalkanoate) (P3HA) homo- or copolymers with different 3-hydroxyalkanoic acids are formed.

The process according to the invention advantageously comprises a prior biomass preparation step, in which the biomass is treated by washing, drying and shredding operations, to give a solid biomass (for example in powder form) containing at least 30% by weight of P3HA, preferably at least 50% by weight of P3HA.

According to a first embodiment of the invention shown in FIG. 1, stream 1 represents the supply of one or more inhibitors into the thermolysis reactor. The polymerization inhibitors used in the process according to the invention are chosen from inhibitors conventionally used in existing industrial processes for the production of α,β-unsaturated carboxylic acids. These include phenol derivatives such as hydroquinone (HQ) and derivatives thereof such as hydroquinone methyl ether (HQME), 2,6-di-tert-butyl-4-methylphenol (BHT) or 2,4-dimethyl-6-tert-butylphenol (Topanol A); phenothiazine and derivatives thereof; nitroxide compounds such as 4-hydroxy-2,2,6,6-tetramethylpiperidin-1-oxyl (4-OH-TEMPO); and amino compounds such as para-phenylenediamine derivatives.

Stream 2 is formed of P3HA previously extracted from biomass. It feeds the thermolysis reactor via a pipe.

Streams 1 and 2 are fed into the thermolysis reactor via a pipe, an endless screw, a conveyor belt or hopper, a pneumatic conveyor, a vibrating conveyor or an extruder. In addition, they may be coupled to a metering device.

According to a preferred embodiment, at least one of said polymerization inhibitors is hydroquinone methyl ether (HQME).

The mass proportion of inhibitor in the mixture with P3HA or with biomass is at least 0.01% and may be up to 90%; preferably, the inhibitor content in said mixture is from 1% to 70%.

When stream 2 is P3HA extracted from its cell membrane, the thermolysis reactor is a solvolysis reactor also used to depolymerize composites or a melt polycondensation type reactor. This reactor is fitted with a heating system and stirring by a pump and external recirculation through an exchanger or a magnetic drive shaft of the double helical belt type.

According to one embodiment, the temperature in the thermolysis reactor is between 20° C. and 250° C., preferably from 150° C. to 200° C. This temperature may also be controlled by means of temperature sensors placed in the reactor. Such moderate heating allows all or some of the mixture to be liquefied while at the same time preventing polymerization of the α,β-unsaturated carboxylic acid.

According to one embodiment, the pressure in the thermolysis reactor is between 3 kPa and 101 kPa, preferably between 15 kPa and 40 kPa.

According to one embodiment, the residence time in the thermolysis reactor is between 0.5 h and 5 h, preferably between 2 h and 4 h.

All or some of stream 3 leaving the thermolysis reactor is sent to an upgrading unit or recycled upstream of the thermolysis reactor.

Stream 5 is fed to a first condenser C1, which cools the gases to a temperature at least 20° C. below the thermolysis reactor temperature, and generates a liquid stream 12 which is recycled into the thermolysis reactor, or which may be purified by crystallization (not shown) prior to recycling into the thermolysis reactor. The non-condensed gases discharged by stream 8 from condenser C1 are condensed in condenser C2 at a temperature at least 20° C. below that of condenser C1. This temperature is adjusted so that the inhibitor mass content in stream 11 is less than 3%.

Stream 11 may be gaseous or liquid after condensation (not shown) before entering the separation column (COL) which will allow recovery of the α,β-unsaturated carboxylic acid with a purity greater than 98.5% at a temperature 5° C. and preferably 10° C. below the bubble point of the feed to this separation column.

Streams 9 and 12 may be totally or partly recycled into the thermolysis reactor or sent to a crystallizing vessel (not represented) for separation and purification, so as to obtain, on the one hand, part of the heavy impurities and the inhibitor, which may be recycled into the thermolysis reactor and, on the other hand, acrylic acid, which may be sent to the separation column (COL) feed.

Condensers C1 and C2 may also be fed with a solution of at least one inhibitor dissolved in α,β-unsaturated carboxylic acid (streams 6 and 7).

The separation column is fitted with a side withdrawal and includes a number of theoretical plates of between 10 and 30, preferably between 20 and 25. This single column operates at a reduced pressure generally of between 3 kPa and 30 kPa, preferably between 9 kPa and 20 kPa.

The separation column consists of any type of plates and/or random internals and/or structured packings available for the rectification of mixtures and suitable for the distillation of polymerizable compounds. It may be a conventional distillation column which may comprise at least one packing, for instance random packing and/or a combination of sections equipped with random and structured packings, and/or plates, for instance perforated plates, fixed valve plates, movable valve plates, bubble plates, or combinations thereof. Preferably, the column is equipped with perforated plates.

The stabilization of the column (stream 17) is generally performed using stabilizers that are well known to those skilled in the art, optionally with injection of air or of oxygen-depleted air (stream 16).

The column is fed in the first bottom quarter of the column, preferably at a plate ranging from plates 1 to 7, preferably from plates 3 to 5.

Stream 4, rich in light compounds, is distilled at the top of the column and upgraded by hydrothermal gasification or at a biological treatment plant after condensation.

The α,β-unsaturated carboxylic acid with a purity >98% is withdrawn in the liquid or gas phase, preferably in the first top third of the separation column, in particular between theoretical plates 1 to 5 located below the column head. Preferably, polymer-grade α,β-unsaturated carboxylic acid is withdrawn as a liquid phase (stream 18). This stream 18 can be further purified by a crystallization operation performed in the crystallizing vessel (CRIS), to achieve a purity of α,β-unsaturated carboxylic acid >99.5% that is compatible with commercial specifications.

According to one embodiment, this last operation affording very high purity α,β-unsaturated carboxylic acid is performed by fractional crystallization. This may be performed in various forms: dynamic crystallization, static crystallization or suspension crystallization.

According to one embodiment, the crystallization is a falling-film process, performed in a multitube exchanger; in practice, each tube is continuously fed at the top with stream 18, a heat-transfer fluid stream. This operation in fact comprises three steps: first, crystallization at a controlled temperature, for example of about 14° C. for acrylic acid, followed by bleeding by a gradual temperature rise of the heat-transfer fluid to remove impurities included in the crystals, and finally melting by a rapid temperature rise above the controlled melting point of the α,β-unsaturated carboxylic acid (about 14° C. for acrylic acid), but preferably below 35-40° C.

The separation column (COL) tailstock is an α,β-unsaturated carboxylic acid stream including the bulk of the heavy impurities, and a large proportion of the inhibitor is totally or partly recycled into the thermolysis reactor.

According to one embodiment, the mass ratio between the side-drawn stream and the column feed stream is between 60% and 95%, preferably between 75% and 90%.

According to one embodiment, the mass ratio between the bottom-drawn stream and the column feed stream is between 5% and 30%, preferably between 5% and 10%.

According to a particular embodiment, the column is equipped with a condenser and a top liquid feed (not shown), which ensures liquid reflux in the column. The reflux ratio, which can be defined as the ratio of the recycling flow rate from the top of the column to the separation column (COL) relative to the side withdrawal flow rate, is between 1 and 3, preferably between 1 and 2, for example equal to 1.2. These conditions allow the best compromise to be reached between the column size/number of separation stages to be used and the energy to be used to ensure efficient distillation.

According to a second embodiment of the invention shown in FIG. 2, stream 1 represents the supply of one or more inhibitors to the thermolysis reactor, as described above.

Stream 2 consists of biomass. It also feeds the pyrolysis reactor, which in this case is a conveyor mixer of the paddle dryer type.

Streams 1 and 2 are fed into the thermolysis reactor via a pipe, an endless screw, a conveyor belt or hopper, a pneumatic conveyor, a vibrating conveyor or an extruder. In addition, they may be coupled to a metering device.

According to a preferred embodiment, at least one of said polymerization inhibitors is hydroquinone methyl ether (HQME).

The mass proportion of inhibitor in the mixture with the biomass is less than 1%.

According to one embodiment, the temperature in the thermolysis reactor is between 20° C. and 250° C., preferably 150° C.-200° C., and the pressure in the reactor is between 3 kPa and 101 kPa, preferably between 15 kPa and 40 kPa.

According to one embodiment, the residence time in the reactor is between 0.5 h and 5 h, preferably between 2 h and 4 h.

Stream 3 leaving the thermolysis reactor is totally or partly sent to a unit for upgrading, by combustion, land spreading or gasification.

According to one embodiment, the residue is mixed with the necessary water and upgraded by hydrothermal gasification.

Stream 5 feeds a first condenser C1, which cools the gases to a temperature below the temperature of the feed plate in the separation column (COL).

The liquid stream 11 enters the separation column at a temperature 5° C. and preferably 10° C. below the bubble point of the column feed.

Condenser C1 may also be fed with a solution of at least one inhibitor dissolved in the α,β-unsaturated carboxylic acid (stream 6).

The separation column is fitted with a side withdrawal and includes a number of theoretical plates of between 10 and 30, preferably between 20 and 25. This single column operates at a reduced pressure generally of between 3 kPa and 30 kPa, preferably between 9 kPa and 20 kPa.

The separation column consists of any type of plates and/or random internals and/or structured packings available for the rectification of mixtures and suitable for the distillation of polymerizable compounds. Preferably, the column is equipped with perforated plates.

The stabilization of the column (stream 17) is generally performed using stabilizers that are well known to those skilled in the art, optionally with injection of air or of oxygen-depleted air (stream 16). The side withdrawal stream can also be stabilized (not shown).

The column is fed in the first bottom quarter of the column, preferably at a plate ranging from plates 3 to 10, preferably from plates 4 to 8.

Stream 4, rich in light compounds, is distilled at the top of the column and upgraded by hydrothermal gasification or at the biological treatment plant.

The α,β-unsaturated carboxylic acid with a purity >98% is withdrawn in the liquid or gas phase, preferably in the first bottom quarter of the separation column, in particular between theoretical plates 10 to 20 located below the column head. Preferably, polymer-grade α,β-unsaturated carboxylic acid is withdrawn as a liquid phase (stream 18). After additional cooling, this stream 18 can be further purified by a crystallization operation in a crystallizing vessel (CRIS), to achieve a purity of α,β-unsaturated carboxylic acid >99.5% that is compatible with commercial specifications.

This last operation affording very high purity α,β-unsaturated carboxylic acid is performed by fractional crystallization.

The separation column (COL) tailstock is an α,β-unsaturated carboxylic acid stream including the bulk of the heavy impurities, and a large proportion of the inhibitor is totally or partly recycled into the thermolysis reactor.

According to one embodiment, the mass ratio between the side-drawn stream and the column feed stream is between 60% and 95%, preferably between 75% and 90%.

According to one embodiment, the mass ratio between the bottom-drawn stream and the column feed stream is between 5% and 30%, preferably between 5% and 10%. According to a particular embodiment, the column COL is equipped with a condenser and a liquid feed at the top (not shown), which ensures liquid reflux in the column. The reflux ratio, which can be defined as the ratio of the recycling flow rate from the top of the column to the separation column (COL) relative to the side withdrawal flow rate, is between 1 and 3, preferably between 1 and 2, for example equal to 1.2.

The examples below illustrate the present invention without, however, limiting the scope thereof.

### EXPERIMENTAL SECTION

The following abbreviations are used in the tables:
AA: acrylic acid
ACOH: acetic acid
H2O: water
PTZ: phenothiazine
HQME: hydroquinone methyl ether
ACETAL: Acetaldehyde
PROH: propanoic acid

Examples 1-4: The aim of this laboratory-scale experiment is to observe the consistency of the residue after a thermolysis operation to determine whether it can be handled after leaving the thermolysis reactor.

Examples 1-4 are performed on biomass containing 60% by weight of P3HP. The α,β-unsaturated carboxylic acid obtained after thermolysis is acrylic acid (AA).

The step of mixing the biomass containing the P3HP with the inhibitor is performed by introducing 2 g of this biomass and 0 or 20 mg of HQME into a 50 mL two-necked round-bottomed flask equipped with a magnetic bar. The medium is stirred using a magnetic stirrer, so as to spread the inhibitor throughout the solid.

At the start of the experiment, the system is placed under the desired pressure, and the round-bottomed flask containing the biomass is then placed in a heating system allowing the desired thermolysis temperature to be established (oil bath or electric heating mantle).

As soon as the thermolysis reactor reaches more than 170° C., AA vapors are formed, condensing predominantly in the side condenser. After 4 h of heating, the formation of AA vapors in the thermolysis reactor tapers off and the experiment is then stopped. The state of fouling and also the consistency of the thermolysis residue are assessed visually at the end of the experiment.

It is found that addition of the inhibitor allows a very hard solid to be transformed into a viscous pasty solid which can be upgraded in the invention.

The operating conditions for tests 1~4 are shown in Table 1.

TABLE 1

| | | Operating pressure | |
|---|---|---|---|
| Test | Inhibitor | (kPa) | Thermolysis residue |
| 1 | HQME | 100 | Viscous pasty solid |
| 2 | HQME | 55 | Viscous pasty solid |
| 3 | HOME | 20 | Viscous pasty solid |
| 4 | NO | 100 | Very hard, tacky solid |

The results in Table 1 demonstrate that the physical state of the residue is dependent on the presence of the inhibitor. The addition of inhibitor enables the residue to become pasty and viscous, whereas it was very hard and tacky without the addition of same. This change in consistency of the residue allows easier extraction of same when it comes to performing the thermolysis continuously.

### Thermogravimetric Analysis Test

The purpose of this test is to provide information regarding the operating conditions required to perform the thermolysis of the P3HA contained in the biomass. In order to better evaluate the degree of thermolysis, this test was performed with P3HP extracted from the biomass.

10 g of pure P3HP were subjected to thermogravimetric analysis. Thermogravimetric analysis is a technique consisting in measuring the variation in mass of a sample as a function of time, for a given temperature. To do this, for each temperature condition, 1 g of P3HP is placed on the apparatus balance while flushing with nitrogen, and the weight loss over time is then recorded. It is found that the mass loss becomes significant and rapid at and above 170° C.

From a thermolysis point of view, the least severe operating conditions would be: T° of 180° C. for a residence time of 3 h, to obtain a degree of cracking of over 95%. Table 2 indicates the temperature conditions and times required to obtain different degrees of cracking, from 10% to complete cracking.

TABLE 2

| | Time required for: | | |
|---|---|---|---|
| T | 10% cracking | 50% cracking | Complete cracking (>95%) |
| 120° C. | >24 h | >24 h | >24 h |
| 130° C. | 14 h | >24 h | >24 h |
| 140° C. | 6 h | 24 h | >24 h |
| 150° C. | 2 h | 8 h | 15 h |
| 160° C. | 1 h 30 | 6 h | 10 h |
| 170° C. | 1 h | 4 h | 8 h |
| 180° C. | 45 min | 2 h | 3 h |
| 190° C. | 30 min | 1 h 15 | 1 h 45 |
| 200° C. | <30 min | 45 min | 1 h |
| 210° C. | <30 min | 40 min | 50 min |
| 220° C. | <30 min | 35 min | 40 min |

The thermolysis reaction temperature of about 180° C. in the process according to the invention is significantly lower than that used in Example 1 of WO 2016/039618.

The tests in the following Examples 5-7 are performed in a laboratory set-up. A two-necked round-bottomed flask equipped with magnetic stirring was used. The side neck of the round-bottom flask is equipped with a thermometer to monitor the reaction temperature. The upper neck of the round-bottom flask is equipped with a separating bridge leading to a water-cooled side condenser, itself leading to a receiver consisting of a second 50 ml round-bottom flask. An additional branch line allows the assembly to be placed under reduced pressure by means of a diaphragm vacuum pump.

### Example 5 (Comparative): Use of a Biomass Containing 60% of P3HP without Addition of Inhibitor, without Catalyst and in the Absence of Inert Gas

2.02 g of biomass containing 60% of P3HP are placed in a 25 mL two-necked round-bottomed flask equipped with magnetic stirring. The round-bottomed flask, equipped with a separating bridge, is pressurized to 20 kPa by means of a diaphragm vacuum pump. The round-bottomed flask is heated at 200° C. for 4 hours. The vapors generated are condensed using a water-cooled side condenser to obtain 1.06 g of acrylic acid, corresponding to an 87% yield. The solid obtained after cracking remains stuck to the walls of the two-necked round-bottomed flask. It is very difficult to remove from the two-necked round-bottomed flask. Solid particles are found in the head of the round-bottomed flask and on the side condenser.

### Example 6 (According to the Invention): Use of a Biomass Containing 60% of P3HP by Addition of 5% of 4-Methoxyphenol (HOME)

2.19 g of biomass containing 60% of P3HP are placed in a 25 mL two-necked round-bottomed flask equipped with magnetic stirring. 0.117 g of HQME is added to the round-bottomed flask and mixed with the biomass. The round-bottomed flask, equipped with a separating bridge, is pressurized to 20 kPa by means of a diaphragm vacuum pump. The round-bottomed flask is heated at 200° C. for 4 hours. The vapors generated are condensed using a water-cooled side condenser to obtain 1.27 g of acrylic acid, corresponding to a 96% yield. The solid obtained after cracking remains compact and readily detaches from the walls of the two-necked round-bottomed flask and is easily broken up with a spatula.

The results of Example 6 show that when the process according to the invention is performed (in the presence of inhibitor, without catalyst, without addition of inert gas), the acrylic acid yield reaches 95%, higher than that obtained in Comparative Example 5.

Furthermore, the presence of the inhibitor allows the residue to be readily removed.

### Example 7: Processes According to the Invention

Simulations were performed on the case of thermolysis of P3HA poly(3-hydroxypropionate) (P3HP) yielding acrylic acid (AA) as α,β-unsaturated carboxylic acid, using the Aspen Tech V12.1 software and Arkema's thermodynamic databases.

The compounds taken into account to represent the gas phase resulting from thermolysis, without the addition of an inert gas, are listed hereinbelow. The percentages are expressed as mass percentages.

### Condensation Process Output as Shown in FIG. 1

Table 3 shows the streams entering and leaving the condensers C1 and C2 when the C1 feed stream 5 is made up of 30% AA and 70% HQME at 200° C. and 26.7 kPa. C1 partially condenses the gases at 150° C. and C2 at 125° C.

The use of two staged condensers makes it possible to obtain an acrylic acid (stream 11) with an inhibitor content of 3%.

In the case studied, streams 6 and 7 were zero.

TABLE 3

| STREAM | | 5 | 12 | 8 | 9 | 11 |
|---|---|---|---|---|---|---|
| Temperature | ° C. | 200.00 | 150.00 | 150.00 | 125.00 | 125.00 |
| Pressure | kPa | 27 | 27 | 27 | 27 | 27 |
| Total mass flow rate | kg/h | 100.00 | 79.50 | 20.50 | 4.96 | 15.54 |
| AA flow rate | kg/h | 30.00 | 12.99 | 17.01 | 1.97 | 15.04 |
| HQME flow rate | kg/h | 70.00 | 66.52 | 3.48 | 2.98 | 0.50 |
| Mass fraction | | | | | | |
| AA | | 0.30 | 0.16 | 0.83 | 0.40 | 0.97 |
| HQME | | 0.70 | 0.84 | 0.17 | 0.60 | 0.03 |

### Distillation Process as Shown in FIG. 1 in the Absence of an Inert Gas.

Table 4 shows the streams entering and leaving the separation column. Said column includes 25 theoretical stages. The feed is introduced at plate 20, the acrylic acid is withdrawn at plate 17, and the light fractions are removed at the top of the column.

In this simulation, streams 16 and 17 were zero.

As can be seen from the composition of the withdrawn stream 18, the acrylic acid has a purity of over 98%.

The acrylic acid recovery balance for the purification process is 98%. Specifically, only the acrylic acid present in stream 4 will be upgraded in hydrothermal gasification.

TABLE 4

| Stream | | 11 | 15 | 4 | 18 |
|---|---|---|---|---|---|
| Temperature | C | 50 | 110 | 30 | 90 |
| Pressure | kPa | 200.00 | 20.00 | 20.00 | 20.00 |
| Mass flow rate | kg/h | 100.00 | 6.00 | 10.00 | 84.00 |
| FORMALDEHYDE | kg/h | 1.32 | 0.00 | 1.31 | 0.01 |
| ACETAL | kg/h | 0.04 | 0.00 | 0.03 | 0.00 |
| ACRO | kg/h | 0.00 | 0.00 | 0.00 | 0.00 |
| H2O | kg/h | 4.87 | 0.00 | 4.56 | 0.31 |
| ACOH | kg/h | 3.15 | 0.00 | 2.19 | 0.95 |
| AA | kg/h | 87.42 | 3.06 | 1.90 | 82.46 |
| PROH | kg/h | 0.28 | 0.02 | 0.00 | 0.26 |
| HQME | kg/h | 2.92 | 2.92 | 0.00 | 0.00 |
| Mass fractions | | | | | |
| FORMALDEHYDE | | 0.01 | 0.00 | 0.13 | 0.000 |
| ACETAL | | 0.00 | 0.00 | 0.00 | 0.000 |
| H2O | | 0.05 | 0.00 | 0.46 | 0.004 |
| ACOH | | 0.03 | 0.00 | 0.22 | 0.011 |
| AA | | 0.87 | 0.51 | 0.19 | 0.982 |
| PROH | | 0.00 | 0.00 | 0.00 | 0.003 |
| HQME | | 0.03 | 0.49 | 0.00 | 0.000 |

### Condensation Process as Shown in FIG. 2 in the Absence of an Inert Gas

Table 5 shows the streams entering and leaving the condenser C1 when the C1 feed stream 5 is made up of 99% AA and 1% HQME at 200° C. and 26.7 kPa. C1 partially condenses the gases at 80° C. The condensation is complete at 80° C., i.e. at a temperature below the bubble point of the column feed plate,

TABLE 5

| Stream | | 5 | 11 |
|---|---|---|---|
| Temperature | C | 200 | 80 |
| Pressure | kPa | 27 | 27 |
| Mass flow rate | kg/h | 100 | 100 |
| AA | kg/h | 99 | 99 |
| HOME | kg/h | 1 | 1 |
| Mass fractions | | | |
| AA | | 0.99 | 0.99 |
| HQME | | 0.01 | 0.01 |

### Distillation Process According to FIG. 2

Table 6 shows the streams entering and leaving the separation column. Said column includes 25 theoretical stages. The feed is introduced at plate 20, the acrylic acid is withdrawn at plate 17, and the light fractions are removed at the top of the column. In this simulation, streams 16 and 17 were zero.

As can be seen from the composition of the withdrawn stream 18, the acrylic acid has a purity of over 98%.

The acrylic acid recovery balance for the purification process is 98.3%. Specifically, only the acrylic acid present in stream 4 will be upgraded in hydrothermal gasification.

TABLE 6

| Stream | Units | 11 | 4 | 15 | 18 |
|---|---|---|---|---|---|
| Temperature | C | 50 | 30 | 98 | 90 |
| Pressure | kPa | 200 | 20 | 20 | 20 |
| Mass flow rate | kg/h | 100.00 | 10.00 | 6.00 | 84.00 |
| FORMALDEHYDE | kg/h | 1.35 | 1.34 | 0.00 | 0.01 |
| ACETAL | kg/h | 0.04 | 0.04 | 0.00 | 0.00 |
| H2O | kg/h | 4.97 | 4.65 | 0.00 | 0.31 |
| ACOH | kg/h | 3.21 | 2.24 | 0.00 | 0.97 |
| AA | kg/h | 89.15 | 1.73 | 4.98 | 82.45 |
| PROH | kg/h | 0.29 | 0.00 | 0.03 | 0.26 |
| HQME | kg/h | 0.99 | 0.00 | 0.99 | 0.00 |
| Mass fractions | | | | | |
| FORMALDEHYDE | | 0.014 | 0.134 | 0.000 | 0.000 |
| ACETAL | | 0.000 | 0.004 | 0.000 | 0.000 |
| H2O | | 0.050 | 0.465 | 0.000 | 0.004 |
| ACOH | | 0.032 | 0.224 | 0.000 | 0.011 |
| AA | | 0.892 | 0.173 | 0.829 | 0.982 |
| PROH | | 0.003 | 0.000 | 0.005 | 0.003 |
| HQME | | 0.010 | 0.000 | 0.166 | 0.000 |

Distillation process according to FIG. 2: Effect of side withdrawal in gas or liquid phase Table 7 shows the side withdrawal streams in the gas or liquid phase. The column includes 25 theoretical stages. The feed is introduced at plate 20, the acrylic acid is withdrawn at plate 17, and the light fractions are removed at the top of the column. In this simulation, streams 16 and 17 were zero. The most favorable configuration for recovering acrylic acid with a purity >98% is that having a liquid phase side withdrawal.

TABLE 7

| Stream | Units | LIQUID PHASE | GAS PHASE |
|---|---|---|---|
| Temperature | C | 90 | 91 |
| Pressure | kPa | 20 | 20 |
| Mass flow rate | kg/h | 84.00 | 84.00 |
| FORMALDEHYDE | kg/h | 0.01 | 0.44 |
| ACETAL | kg/h | 0.00 | 0.01 |
| H2O | kg/h | 0.31 | 1.78 |
| ACOH | kg/h | 0.97 | 1.58 |
| AA | kg/h | 82.45 | 79.93 |
| PROH | kg/h | 0.26 | 0.26 |
| HQME | kg/h | 0.00 | 0.00 |
| Mass fractions | | | |
| FORMALDEHYDE | | 0.000 | 0.01 |
| ACETAL | | 0.000 | 0.00 |
| H2O | | 0.004 | 0.02 |
| ACOH | | 0.011 | 0.02 |
| AA | | 0.982 | 0.95 |
| PROH | | 0.003 | 0.00 |
| HQME | | 0.000 | 0.00 |

1. A process for manufacturing a biobased α,β-unsaturated carboxylic acid, said process comprising the following steps:
mixing a biomass containing a poly(3-hydroxyalkanoate) (P3HA), or solid P3HA, with at least one polymerization inhibitor to obtain a biomass/inhibitor mixture or a P3HA/inhibitor mixture, respectively;
subjecting said biomass/inhibitor mixture or P3HA/inhibitor mixture, without the addition of catalyst and without the addition of an inert gas, to a thermolysis step, resulting in said α,β-unsaturated carboxylic acid in the vapor phase and a molten or solid residue;
condensing thermolysis gases resulting from thermolysis in one or more steps, and then feeding the resulting condensed thermolysis gases to a distillation column;
fractionating the thermolysis gases into light products, heavy products recycled to the thermolysis reactor, and α,β-unsaturated carboxylic acid with a purity of greater than 98%, which can be crystallized to reach a purity greater than 99.5%; and
treating the solid-phase residue or recycling it to the thermolysis reactor inlet.

2. The process as claimed in claim 1, wherein the following steps are performed sequentially or simultaneously, and wherein:
mixing the biomass containing a poly(3-hydroxyalkanoate) (P3HA), or the solid P3HA, with the at least one polymerization inhibitor by introducing the biomass (in powder form) or the P3HP and the at least one polymerization inhibitor into a conveyor mixer comprising several endless screws actuated in a sheath or directly in a reactor known as a thermolysis reactor) by means of a pipe or by an endless screw-type conveyor;
the thermolysis step is carried out at a thermolysis temperature and controlled thermolysis pressure in a system suitable for treating the biomass/inhibitor mixture or P3HA/inhibitor mixture to generate a vapor phase and a solid phase residue;
the method further comprising:
separation of the vapor phase and the solid phase residue in a gas/liquid separator;
treating the solid phase residue by land spreading, combustion, hydrothermal gasification or recycling upstream of the pyrolysis reactor;
staged condensation of the gas phase, performed by adjusting successive condensation temperatures using one or more condensers in series, and separation of the obtained gas and liquid phases containing acrylic acid and contaminants, which may be recycled back into the reactor or sent to the purification system;
treating the condensed phase to obtain acrylic acid is carried out in one or more distillation columns allowing, on the one hand, the separation of the acrylic acid from the products heavier than same and, on the other hand, the production of products lighter than same; and
purifying the acrylic acid via a liquid/solid separation method or by a gas/liquid separation method.

3. The process as claimed in claim 1, wherein the biomass is pretreated by means of washing, drying or shredding operations to yield a biomass containing at least 30% by weight of P3HA.

4. The process as claimed in claim 1, wherein the poly(3-hydroxyalkanoate) contains a 3-hydroxypropionate unit and at least one of the α,β-unsaturated carboxylic acids produced is acrylic acid.

5. The process as claimed in claim 1, wherein the poly(3-hydroxyalkanoate) is poly(3-hydroxypropionate) and the α,β-unsaturated carboxylic acid produced is acrylic acid.

6. The process as claimed in claim 1, wherein the poly(3-hydroxyalkanoate) contains a 3-hydroxybutyrate unit and at least one of the α,β-unsaturated carboxylic acids produced is crotonic acid.

7. The process as claimed in claim 1, wherein the poly(3-hydroxyalkanoate) is poly(3-hydroxybutyrate) and the α,β-unsaturated carboxylic acid produced is crotonic acid.

8. The process as claimed in claim 1, wherein the poly(3-hydroxyalkanoate) contains a 3-hydroxyisobutyrate unit and at least one of the α,β-unsaturated carboxylic acids produced is methacrylic acid.

9. The process as claimed in claim 1, wherein the poly(3-hydroxyalkanoate) is poly(3-hydroxyisobutyrate) and the α,β-unsaturated carboxylic acid produced is methacrylic acid.

10. The process as claimed in claim 1, wherein the at least one polymerization inhibitor(s) are compounds inhibitor is chosen from phenol derivatives, phenothiazine derivatives, nitroxide derivatives or and para-phenylenediamine derivatives.

11. The process as claimed in claim 10, wherein the at least one polymerization inhibitors is hydroquinone methyl ether.

12. The process as claimed in claim 2, wherein the thermolysis reactor is chosen from: a conveyor, a conveyor mixer, a dryer, a rotating drum and/or a set of heating plates.

13. The process as claimed in claim 12, wherein the thermolysis reactor is a solvolysis reactor also used to depolymerize composites or a melt polycondensation type reactor.

14. The process as claimed in claim 12, wherein the thermolysis reaction is performed at a temperature of between 150° C. and 200° C.

15. The process as claimed in claim 1, wherein the thermolysis step is performed for two to four hours.

16. The process as claimed in claim 1, wherein the thermolysis pressure is between 15 kPa and 40 kPa.

17. The process as claimed in claim 1, wherein separation of the vapor phase and the solid phase residue is carried out in a separation column, and wherein the separation column is fitted with a side withdrawal and includes a number of theoretical plates of between 10 and 30.

18. The process as claimed in claim 17, wherein the separation column operates under a reduced pressure between 3 kPa and 30 kPa.

19. The process as claimed in claim 17, wherein withdrawal from the separation column is performed in the liquid phase.

20. The process as claimed in claim 12, wherein the mixing the biomass containing a poly(3-hydroxyalkanoate) (P3HA), or the solid P3HA, with the at least one polymerization inhibitor and the thermolysis step may be performed consecutively or simultaneously.
